(19) United States
(12) Patent Application Publication
(10) Pub. No.: US 2022/0133044 A1
(43) Pub. Date: May 5, 2022

(54) PHYSICAL MODALITY TREATMENT CHAIR

(71) Applicant: Archer Lewis IRBY, Brooklyn, NY (US)

(72) Inventor: Archer Lewis IRBY, Brooklyn, NY (US)

(21) Appl. No.: 17/090,856

(22) Filed: Nov. 5, 2020

(51) Int. Cl. A47C 1/032; A61H 1/00; A61H 1/02; A61N 1/36

(52) U.S. Cl. CPC A47C 1/03205; A47C 1/03255; A61H 1/006; A61H 1/0218; A61N 1/36014; A61H 2201/1633; A61H 2201/5064; A61H 2205/081; A61H 2201/1626; A61H 2201/0207; A61H 2201/10; A61H 2001/0203; A61H 2203/0431

(57) ABSTRACT

A therapeutic chair is disclosed with moving parts that permit a user to implement a regimen of physical modality treatment, including non-axial vertebral decompression, flexion extension, distraction, rotation, and lateral flexion.

### BACKGROUND OF THE INVENTION

Low back pain contributes to 95% of Emergency Room (ER) Emergency Department (ED) visits that involve musculoskeletal disorders. A 2010 study by National Institutes of Health (NIH), found that low back pain related disorders caused 2.63 million annual ED visits in the US. Imaging is expensive and drives up the cost of healthcare. A 2006 study found that 30.5% of low back pain patients had a plain radiograph; and 9.6% had a CT or MRI. Drug treatment of low back pain is problematic. Of medications either administered in the ED or prescribed at discharge, the most frequently used classes were opioids, followed by non-steroidal anti-inflammatory drugs, and muscle relaxants. There continues to be an unmet need for treatment of low back pain at acceptable cost that reduces reliance on drug regimens.

The “McKenzie Method” is probably the most recognizable method utilized by most therapists who treat low back pain, including physical therapists, chiropractors, physiatrists, pain rehab doctors, as well as by orthopedic surgeons before performing back surgery. The McKenzie method is a comprehensive approach to the spine based on sound principles and fundamentals and when applied correctly, results are almost guaranteed. However, good results depend on patient compliance. The investment of time and money to learn and maintain a physical regimen poses challenges for many low-back pain sufferers.

Chairs, particularly office chairs, have been developed that are capable of all types of movement, in an effort to reduce pain and improve the posture of those destined to sit in them day after day for hours at a time. Thus, a rotating seat, a reclining backrest, movable lumbar support, forward and rear tilt of a seat rest, with locked and free positions, are all by now typical features for an office chair. U.S. Pat. Nos. 6,003,949, 6,244,659, 7,794,017, 9,016,790 and U.S. Patent Application Publication No. 2009/0195040 are incorporated by reference for their teaching of conventional means for attaching a seat member and a back member to a frame, as well as means to cause different parts of a chair to pivot, slide and rotate with respect to one another. However, an office chair designed for achieving a comfortable or correct position is not configured for the simultaneous activation of spinal positions that will yield a physical modality treatment familiar to the physical therapist, osteopath or orthopedist.

Devices that have been developed for use in the chiropractor's offices, including various traction devices, have not been configured to implement non-axial decompression, flexion, extension, distraction, rotation, and lateral flexion, in a convenient manner which lends itself to self-use of the devices. These modalities are often recommended for the treatment of lower back pain and related conditions and at present no suitable apparatus is known or commercially available for achieving these results. Various exercise apparatuses have been proposed, and these likewise are not adapted to physical modality treatment of lower back pain and related symptoms.

### SUMMARY OF THE INVENTION

Thus, an object of the invention is to provide a device, usable at home, that delivers treatment to a subject's neuromusculoskeletal system and assists in skeletal structural alignment, which when used in a proper regimen of treatment may alleviate back pain and other associated symptoms.

In one aspect, a chair according to the invention allows the operator to achieve McKenzie-type extensions, allowing an individual to perform their exercises at any given time, in some embodiments even while sitting at their desk.

These and other objects of the invention are achieved, according to certain embodiments of the invention, with a device for physical modality treatment, comprising: a frame member; a planar seat member having a front and rear portion connected to the frame member and oriented in a generally horizontal plane, adapted to contact a subject's buttocks in a seated position; a planar back member connected to the frame and oriented in a generally vertical plane, and adapted to support a subject's back in a seated position; a slide member connected to said frame, adapted to translate said planar seat member in a horizontal direction away from the planar back member; a seat pivot adapted to pivot said planar seat member about an axis out of the horizontal plane to rotate the planar seat member and raise a rear portion of the planar seat member; a back pivot adapted to pivot said planar back member out of the vertical plane to recline the back member, forming an angle with the vertical; wherein the slide, the seat pivot and the back pivot are adapted to translate and pivot the seat member and pivot the back member simultaneously, to effect physical modality treatment of the subject.

For example, an appropriate regimen of treatment using the chair may comprise non-axial vertebral decompression. The chair may be used to implement flexion, extension, distraction, rotation, and lateral flexion of a subject's spine.

The invention may also be characterized as a method for providing physical modality treatment including at least one of flexion, distraction, extension, non-axial vertebral decompression, rotation and lateral flexion, to a subject in need thereof, comprising: seating the subject in a chair comprising a frame member, a planar seat member connected to said frame member and oriented in a generally horizontal plane, and a planar back member connected to said frame and oriented in a generally vertical plane; such that the subject's buttocks contact the planar seat member and the subject's back contacts the planar back member while the subject is seated in the chair; simultaneously translating the planar seat member on the frame in a horizontal direction away from the planar back member; pivoting the planar seat member about an axis in the horizontal plane to rotate the planar seat member, thereby raising a rear portion of the planar seat member to lower the subject's knees with respect to a reference axis; and pivoting said planar back member in a reclining direction to effect physical modality treatment of the subject.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts a device for physical modality treatment, also referred to herein as the “chair,” in a starting or neutral position.

FIG. 2 is a detail showing the relative movement of seat and back portions of the chair of FIG. 1 to achieve extension and/or flexion of a subject's spine.

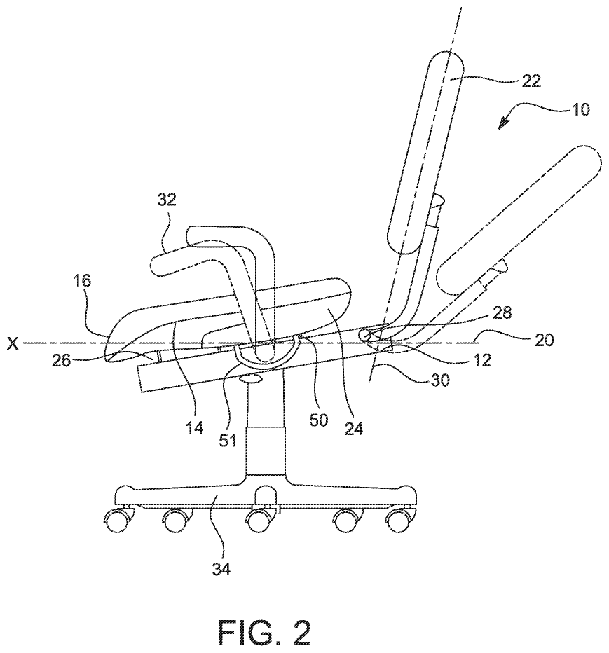

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

A treatment chair according to the invention may be referred to as an “ETS Chair.” The acronym is derived from extension and treatment while sitting. The ETS Chair addresses problems resulting from identified postural mal-positions, dysfunctions, and derangements identified by the clinician of subjects suffering from low back pain, as well as cervical and thoracic spine pain. The description herein addresses use of the chair to alleviates a subject's pain. The ETS Chair targets end range stresses in the low back, from scarring and nerve root involvement due in part to shortened structures, which affect motion segments.

Utilizing the ETS Chair, symptoms of degenerative pain syndromes such as sciatica, facet syndrome, spinal stenosis, subluxation or mal-alignment syndrome, are centralized to the low back. For example, successful treatment(s) are achieved by the individual when pain into the gluteus or pain that radiates down the legs becomes localized to the person's low back.

The ETS Chair creates a traction (joint separation in the low back vertebra) when the chair reclines and the seat moves slightly forward axially, slightly 1-5 degrees downward, and the rear portion of seat extends upwards 1-15 degrees. The seat can also rotate, which helps open the degenerative foramina canals. These functionalities help to achieve comfortable treatment positions, controlled either manually or mechanically by the hand rests attached to a cylinder tube on the chair and seat. The cylinder may be provided with a rotational locking device that when pulled outward and rotated allows the rear of the seat to move upwards and lock into place. The chair may be provided with a spring and using the handles the subject may rock backward and forward pumping the disc, creating separation of locked joints thus alleviating syndromes associated with radiating pain into the lower extremities.

Velcro cushioned attachments can be attached for added extension on the rear of the chair to create more joint separation in the lumbar spine. This provides more patient comfort level adding to therapeutic relief. The manual component may be controlled by the arm rest attached to the ball level, allowing more control by the person to extend backward and forward for a more controlled therapeutic relief. The chair can be equipped with heat and/or electrical stimulation to loosen tight supporting muscles of the low back.

As described below, Bluetooth functionality may be provided to track low back outcome assessment, patient optimal angle for therapeutic relief, and to track regimen compliance, (e.g., 2×/wk for 6 weeks, 3-4 wk for 4 weeks, etc.). An app or instructional interface may be provided for demo purposes, to calculate the time spent sitting and send reminders to the subject to get up from a sitting position, move around to exercise, adjust sitting positions, etc.

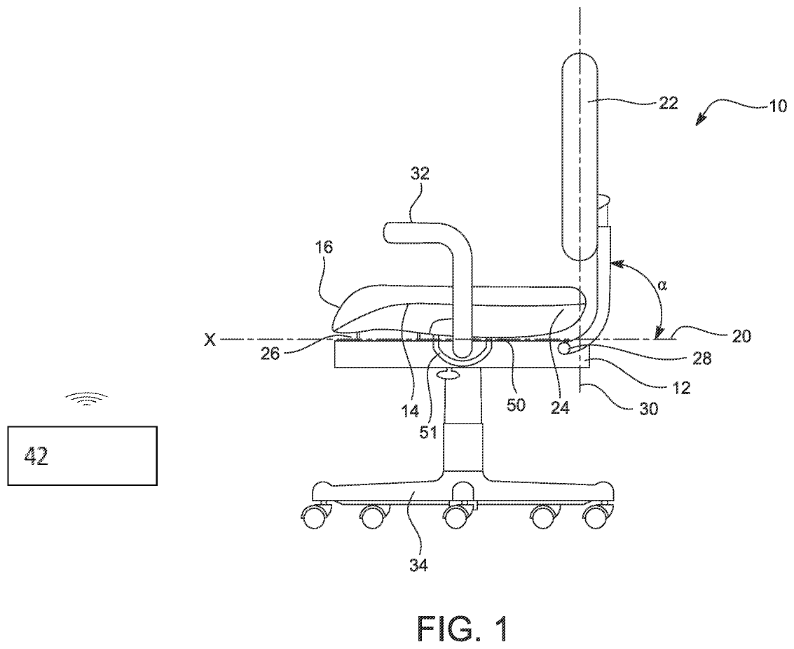

FIG. 1 depicts an embodiment of a device 10 for physical modality treatment in a neutral or starting position according to embodiments of the invention, comprising: frame member 12; and planar seat member 14, having front portion 16 and rear portion 24, and oriented in a generally horizontal plane indicated at 20. It will be appreciated that the seat can have any desired contour and might form an angle of about +/−5° and it would still be in a “generally horizontal” plane. The generally horizontal plane is thus a starting or reference point. Generally horizontal simply means that the seat is adapted to contact a subject's buttocks in a seated position like a normal chair. The range of motion of the seat is described with reference to this starting position, which may be referred to as a reference horizontal axis.

Planar back member 22 is connected to frame 12 and is oriented in a generally vertical plane. As with seat member 14, “generally vertical” back member may include a contour and may recline slightly in the neutral or starting position. The range of motion is described with reference to the starting position which may be referred to as a reference vertical axis 30. Slide member 50 connected to frame 12 is adapted to translate planar seat member 14 in a horizontal direction away from the planar back member. Seat pivot 51 is adapted to pivot planar seat member out of the horizontal plane to rotate the planar seat member and raise rear portion 24 of planar seat member 14. Back pivot 28 is adapted to pivot planar back member 22 out of the vertical plane to recline back member 22, forming an angle with the vertical reference axis 30. Slide member 26, seat pivot 26 and back pivot 28 are adapted to translate and pivot the seat member 16 and pivot back member 22 simultaneously, to effect physical modality treatment of the subject.

The simultaneous movement of the seat and back is adapted to provide at least one of flexion, extension, distraction, rotation, and lateral flexion to the subject. In embodiments, the back member is adapted to form an angle of 1 degrees to 15 degrees with respect to a reference vertical axis, and wherein the seat pivot is adapted to raise the back portion of the seat to form an angle of between 1 degrees and 15 degrees with respect to a reference horizontal axis. In the embodiment shown, frame member 12 pivots with seat member 14. In other embodiments, the frame may remain fixed and the seal member pivots to rotate the front of the planar seat member 16 forward and downward.

In embodiments, the back pivot 28 and the seat pivot 51 both comprise at least one locked position, accessible (for example) by levers connected to arm(s) 32 adapted so that the position of the planar seat member and the planar back member do not change from the locked position until automatically or manually released. In this way, the chair may be used with a treatment regimen, whereby the subject locates a position offering suitable flexion and is able to lock and release that position to complete a series. Thus, a locked position may be settable by a subject seated in the device. The construction of suitable locking and release mechanisms may be left to the skill of the ordinary artisan.

Taking the fixed position one step further in connection with a treatment regimen, a data recorder 42 may record the locked positions of the seat member and the back member, and details related to the subject and a session during which the device is being used.

To provide lateral flexion, planar seat member 14 may be connected to the frame by a rotating member adapted to rotate the planar seat member 90 degrees in either direction in the horizontal plane. The ability of a seat member to rotate is of course not new, but the combination with the seat moving away from the back member and tilting forward while the back reclines to provide lateral flexion is not taught or suggested in the prior art. The left and right rotational positions, at their furthest point, may be subject to locking and automated recording as described above in connection with the core pivoting and sliding actions.

In embodiments, the device further comprises a movable lumbar member adapted to contact a lumbar region of the subject's back, which may be formed as part of the rear portion 24 of seat member 14. In embodiments, movable lumbar member forms a part of seat 14, and is adapted to pivot with respect to seat member 14 in an amount less than 10°, and in embodiments up to 5° simultaneously with the translating and pivoting of the seat member and pivoting of the back member. A similar function could be provided by a separate element located toward the bottom of back member 22.

At least one of the seat member 14, the back member 22, and the lumbar member (shown as part of rear seat portion 24) may be adapted to provide heat and/or electrical stimulation to a subject seated in the chair.

With the device described treatment may be prescribed for a subject to provide physical modality treatment including at least one of flexion, distraction, extension, non-axial vertebral decompression, rotation and lateral flexion. To perform the method of treatment, a subject is seated in a chair as described above, comprising a frame member, a planar seat member connected to said frame member and oriented in a generally horizontal plane, and a planar back member connected to said frame and oriented in a generally vertical plane; such that the subject's buttocks contact the planar seat member and the subject's back contacts the planar back member while the subject is seated in the chair. The subject's spine is treated by simultaneously translating the planar seat member on the frame in a horizontal direction away from the planar back member; pivoting the planar seat member about an axis in the horizontal plane to rotate the planar seat member, thereby raising a rear portion of the planar seat member to lower the subject's knees with respect to a reference axis; and pivoting said planar back member in a reclining direction.

These steps may be conducted to reach a locked position such that the seat member forms an angle in a range of 1 to 15 degrees with respect to the reference horizontal axis, and the back member is pivoted to reach a locked position such that the back member forms an angle in a range of 1 to 15 degrees with respect to a reference vertical axis and then repeated according to a treatment regimen. A different or additional treatment protocol may comprise rotating the seat member up to 90 degrees in either direction in the horizontal plane when said seat member and back member are in the locked positions.

Means may be provided for automatically logging position data of the seat member and the back member during a treatment session and associating the position data with a treatment session of the subject.

The method may comprise simultaneously pivoting the planar seat member to raise a rear portion of the seat member to a first locked position, reclining the planar back member to a second locked position, and translating the seat member away from the back member to a third locked position; releasing said first, second and third locked positions, and repeating said pivoting, reclining and translating motions according to a predetermined regimen.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A device for physical modality treatment, comprising:
a frame member;
a planar seat member having a front and rear portion connected to said frame member and oriented in a generally horizontal plane, adapted to contact a subject's buttocks in a seated position;
a planar back member connected to said frame and oriented in a generally vertical plane, and adapted to support a subject's back in a seated position;
a slide member connected to said frame, adapted to translate said planar seat member in a horizontal direction away from the planar back member;
a seat pivot adapted to pivot said planar seat member about an axis out of the horizontal plane to rotate the planar seat member and raise a rear portion of the planar seat member;
a back pivot adapted to pivot said planar back member out of the vertical plane to recline the back member, forming an angle with the vertical; wherein
the slide, the seat pivot and the back pivot are adapted to translate and pivot the seat member and pivot the back member simultaneously, to effect physical modality treatment of the subject.

2. The device according to claim 1, wherein said physical modality treatment comprises non axial vertebral decompression.

3. The device according to claim 2, wherein said physical modality treatment further comprises at least one of flexion, extension, distraction, rotation, and lateral flexion.

4. The device according to claim 1, wherein the back pivot is adapted to rotate the back member to form an angle of 1 degrees to 15 degrees with respect to a reference vertical axis, and wherein the seat pivot is adapted to raise the back portion of the seat to form an angle of between 1 degrees and 15 degrees with respect to a reference horizontal axis.

5. The device according to claim 4, wherein the back pivot and the seat pivot both comprise at least one locked position, adapted so that the position of the planar seat member and the planar back member do not change from the locked position until automatically or manually released.

6. The device according to claim 5, wherein at least one locked position is adapted for assisted or automated release.

7. The device according to claim 3, wherein at least one locked position is settable by a subject seated in the device.

8. The device according to claim 5, further comprising a data recorder, recording at least the locked positions of the seat member and the back member, and details related to the subject and a session during which the device is being used.

9. The device according to claim 5 wherein the planar seat member is connected to the frame by a rotating member adapted to rotate the planar seat member 90 degrees in either direction in the horizontal plane.

10. The device according to claim 9, wherein the planar seat member is adapted to lock in at least one left and right rotational positions.

11. The device according to claim 1, further comprising a movable lumbar member adapted to contact a lumbar region of the subject's back.

12. The device according to claim 11, wherein the movable lumbar member is adapted to move simultaneously with the translating and pivoting of the seat member and pivoting of the back member.

13. The device according to claim 12, wherein at least one of the seat member, the back member, and the lumbar member is adapted to provide heat and/or electrical stimulation to a subject seated in the chair.

14. The device according to claim 1, wherein the seat member is adapted to rotate out of the horizontal plane to lower or raise a lateral portion of the seat member up to 5 degrees.

15. A method for providing physical modality treatment including at least one of flexion, distraction, extension, non-axial vertebral decompression, rotation and lateral flexion, to a subject in need thereof, comprising:
seating the subject in a chair comprising a frame member, a planar seat member connected to said frame member and oriented in a generally horizontal plane, and a planar back member connected to said frame and oriented in a generally vertical plane; such that the subject's buttocks contact the planar seat member and the subject's back contacts the planar back member while the subject is seated in the chair;
simultaneously translating the planar seat member on the frame in a horizontal direction away from the planar back member;
pivoting the planar seat member about an axis in the horizontal plane to rotate the planar seat member, thereby raising a rear portion of the planar seat member to lower the subject's knees with respect to a reference axis; and
pivoting said planar back member in a reclining direction;
to effect physical modality treatment of the subject.

16. The method according to claim 15, wherein the planar seat member is pivoted to reach a locked position such that the seat member forms an angle in a range of 1 to 15 degrees with respect to the reference horizontal axis, and the back member is pivoted to reach a locked position such that the back member forms an angle in a range of 1 to 15 degrees with respect to a reference vertical axis.

17. The method according to claim 16, further comprising rotating the seat member up to 90 degrees in either direction in the horizontal plane when said seat member and back member are in the locked positions.

18. The method according to claim 15, further comprising automatically logging position data of the seat member and the back member and associating the position data with a treatment session of the subject.

19. The method according to claim 15, wherein releasing from the locked position is performed by the subject from a seated position in the chair.

20. The method according to claim 15, comprising simultaneously pivoting the planar seat member to raise a rear portion of the seat member to a first locked position, reclining the planar back member to a second locked position, and translating the seat member away from the back member to a third locked position; releasing said first, second and third locked positions, and repeating said pivoting, reclining and translating motions according to a predetermined regimen.
